(19) United States
(12) Patent Application Publication
(10) Pub. No.: US 2018/0325594 A1
(43) Pub. Date: Nov. 15, 2018

(54) METHODS OF TREATING SKIN CONDITIONS USING PLASMONIC NANOPARTICLES

(71) Applicant: SEBACIA, INC., Duluth, GA (US)

(72) Inventor: Dilip PAITHANKAR, Wayland, MA (US)

(21) Appl. No.: 15/775,712

(22) PCT Filed: Nov. 14, 2016
(86) PCT No.: PCT/US2016/061827

§ 371 Date: May 11, 2018

Related U.S. Application Data
(60) Provisional application No. 62/254,760, filed on Nov. 13, 2015.

(51) Int. Cl. A61B 18/20; A61K 33/24; A61K 9/14; A61P 17/00; A61P 31/00

(52) U.S. Cl. CPC A61B 18/203; A61K 33/24; A61B 2018/0047; A61P 17/00; A61P 31/00; A61K 9/14

(57) ABSTRACT

Methods, and materials useful in such methods, of treating certain skin conditions are described. In brief, the methods impregnate portions of the skin needing treatment with plasmonic materials. Thereafter, surface plasmons are generated on the surface of these plasmonic materials by irradiating the treated skin with near infrared light that is absorbed by the plasmonic materials in the skin.

### FIELD OF THE INVENTION

The field of the invention is plasmonic materials, including use of plasmonic nanoparticles in therapeutic procedures.

### DESCRIPTION OF THE RELATED ART

Laser treatment of the skin is widely known and has been highly touted by skin care professionals for therapeutic purposes. Potential uses for laser skin therapy include cosmetic laser applications, laser ablation of cancerous cells in cancer patients, and laser ablation of damaged tissue in burn victims.

### SUMMARY

Mammals, including humans, can develop certain skin conditions that call for treatment. Such skin conditions include: acne; acne scars; actinic keratosis; age spots; discoloration (blotchy complexion, uneven skin tone); seborrheic dermatitis; hidradenitis suppurativa; hyperhidrosis; melasma; molluscum contagiosum; pityriasis rosea; psoriasis; rosacea; tinea versicolor; warts; tattoo removal; and fungal skin infections. These skin conditions may be caused by a skin appendage functioning in an undesired manner, microbial infections, solar radiation, or other causes. Mammals (including humans) typically have a unique set of genes, and as a result, may respond to any treatment regime in a manner different from how another individual responds. Consequently dermatologists need additional ways of treating such skin conditions.

Many of these skin conditions that need treatment would benefit from improved methods to specifically thermally ablate certain cells in the subject's dermis and/or epidermis without impairing the surrounding cells.

Some acne vulgaris results from obstruction of the pilosebaceous unit, consisting of the hair shaft, hair follicle, sebaceous gland and erector pili muscle. Such an obstruction may lead to accumulation of sebum oil produced from the sebaceous gland and the subsequent colonization of bacteria within the follicle. Microcomedones formed as a result of accumulated sebum progress to non-inflamed skin blemishes (white/blackheads), or to skin blemishes which recruit inflammatory cells and lead to the formation of papules, nodules and pus-filled cysts. The sequelae of untreated acne vulgaris often include hyperpigmentation, scarring and disfiguration, as well as significant psychological distress. Therefore, acne treatments seek broadly to reduce the accumulation of sebum and microorganisms within follicles and the sebaceous gland.

Methods involving light and lasers are promising for the treatment skin disorders, but are still insufficiently effective. Ultraviolet (UV)/blue light is approved by the FDA for the treatment of mild to moderate acne only, due to its anti-inflammatory effects mediated on skin cells (keratinocytes), potentially through the action of endogenous porphyrin photosensitizers within follicles. However, high intensity energies (50-150 J/cm²) are required to damage sebaceous gland skin structures, and transdermal porphyrin penetration leads to off-target side-effects which include sensitivity to light, pain, inflammation, hyper/hypo-pigmentation, and permanent scarring. Additionally, most light wavelengths are largely unable to penetrate the skin, which acts as a filter and prevents the transmission of most wavelengths other than those between about 750 nm and about 12 nm (these wavelengths that are able to penetrate the skin better than other wavelengths are generally identified as near infrared or “NIR”).

The present invention proposes to thermally ablate only certain specified cells in the dermis and/or epidermis, without ablating surrounding and other cells. This selective thermal ablation can be achieved by putting a plasmonic material that will generate a surface plasmon into the dermis and/or epidermis in close proximity to the cells that are to be ablated.

The present invention, in certain embodiments, provides new compositions and methods useful in the targeted thermoablation of target cells for treating certain skin conditions.

In some embodiments, the composition comprises plasmonic nanoparticles that are activated by exposure to energy delivered from a nonlinear excitation surface plasmon resonance source to cell in the target tissue region. In further or additional embodiments, provided herein is a composition wherein a substantial amount of the plasmonic particles present in the composition comprise nanostructures geometrically-tuned to have a local surface plasmonic resonance in response to NIR radiation. In certain embodiments, provided herein is a composition wherein plasmonic particles comprise any geometric shape currently known or to be created that absorb light and generate plasmon resonance at a desired wavelength, including nanoplates, solid nanoshells, hollow nanoshells, nanorods, nanorice, nanospheres, nanofibers, nanowires, nanopyramids, nanobipyramids, nanoprisms, nanostars or a combination thereof. In yet additional embodiments, described herein is a composition wherein the plasmonic particles comprise silver, gold, nickel, copper, titanium, palladium, platinum, chromium, or titanium nitride.

In some embodiments, provided herein is a composition comprising a cosmetically acceptable carrier that comprises an additive, a colorant, an emulsifier, a fragrance, a humectant, a polymerizable monomer, a stabilizer, a solvent, or a surfactant. In one embodiment, provided herein is a composition wherein the surfactant is selected from the group consisting of: sodium laureth 2-sulfate, sodium dodecyl sulfate, ammonium lauryl sulfate, sodium octech-1/deceth-1 sulfate, lipids, proteins, peptides or derivatives thereof. In one embodiment, provided is a composition wherein a surfactant is present in an amount between about 0.1 and about 10.0% weight-to-weight of the carrier. In yet another embodiment, the solvent is selected from the group consisting of water, propylene glycol, alcohol, hydrocarbon, chloroform, acid, base, acetone, diethyl-ether, dimethyl sulfoxide, dimethylformamide, acetonitrile, tetrahydrofuran, dichloromethane, and ethylacetate. Preferably, the composition comprises plasmonic particles that have an optical density of at least about 1 O.D. at a NIR wavelength.

In further or additional embodiments, described herein is a composition wherein plasmonic particles comprise a coating, wherein the coating does not substantially adsorb to skin of a mammalian subject, and wherein the coating comprises polyethylene glycol (PEG), silica, silica-oxide, polyvinylpyrrolidone, polystyrene, silica, silver, polyvinylpyrrolidone (PVP), cetyl trimethylammonium bromide (CTAB), citrate, lipoic acid, short chain polyethylenimine (PI) and branched polyethylenimine, reduced graphene oxide, a protein, a peptide, or a glycosaminoglycan such as keratan sulfate, and chondroitin sulfate. A preferred PEG coating comprises 5,000 MW PEG moieties.

It is further preferred that the coating on the plasmonic material is at least about 5 nm thick. Generally, the coating is less than about 100 nm thick. It is further preferred that the coating layer is between about 5 and 50 nm. It is further preferred that the coating does not chemically interact with the dermis or epidermis.

Preferred target regions to treat skin conditions include hair follicles, hair follicle infundibulum, sebaceous glands and components thereof, apocrine sweat glands eccrine sweat glands, and oily glands. Within such skin appendiges, the target may include a bulge, a bulb, a stem cell, a stem cell niche, a dermal papilla, a cortex, a cuticle, a hair sheath, a medulla, a pylori muscle, a Huxley layer, or a Henle layer.

In another aspect of the present invention provides a method of performing targeted thermal ablation of tissue. For example, in one embodiment, provided is a method for performing targeted ablation of a tissue to treat a mammalian subject in need thereof, comprising the steps of i) topically administering to a skin surface of the subject the composition of claim 1; ii) providing penetration means to redistribute the plasmonic particles from the skin surface to a component of dermal tissue; and iii) causing irradiation of the skin surface by light. In further or additional embodiments, provided is a method wherein the light source comprises excitation of mercury, xenon, deuterium, or a metal-halide, phosphorescence, incandescence, luminescence, light emitting diode, or sunlight. In still further or additional embodiments, provided is a method wherein the penetration means comprises high frequency ultrasound, low frequency ultrasound, massage, iontophoresis, high pressure air flow, high pressure liquid flow, vacuum, pre-treatment with fractionated photothermolysis or dermabrasion, or a combination thereof. In still further embodiments, provided is a method wherein the irradiation comprises light having a wavelength of light between about 200 nm and about 10,000 nm, a fluence of about 1 to about 100 joules/cm², a pulse width of about 1 femptosecond to about 1 second, and a repetition frequency of about 1 Hz to about 1 THz.

In a further aspect, provided herein is a composition comprising a cosmetically acceptable carrier, an effective amount of sodium dodecyl sulfate, and a plurality of plasmonic nanoparticles in an amount effective to induce thermal damage in a target tissue region with which the composition is topically contacted, wherein the nanoparticles have an optical density of at least about 1 O.D. at a resonance wavelength of about 810 nanometers or 1064 nanometers, wherein the plasmonic particles comprise a silica coating from about 5 to about 35 nanometers, wherein the acceptable carrier comprises water and propylene glycol.

In yet another aspect, provided is a system for laser ablation of hair or treatment of acne comprising a composition and a source of plasmonic energy suitable for application to the human skin.

The process of the present invention puts plasmonic material in the vicinity, i.e., within about 100 microns of the condition to be treated, preferable within about 50 microns, and more preferably within about 10 nm. Inducing a surface plasmon on the plasmonic materials produces localized heating in the vicinity of particle or particles of 20-200 nm, 200 nm-2 μm, 2-20 μm, 20-200 μm, 200 μm-2 mm.

The plasmonic nanoparticles used in the present invention can have substantially any geometry including nanoplates, solid nanoshells, hollow nanoshells, nanorods, nanorice, nanospheres, nanofibers, nanowires, nanopyramids, nanobipyramids, nanobipyramids, nanoprisms, nanostars or a combination thereof. It is preferred that the nanoparticle has an aspect ratio (length divided by thickness) of at least about 2 but less than about 1000. A preferred range of nanoparticle aspect ratios runs from about 3.5, but less than about 20. Nanoparticles having such aspect rations general include nanorods, metallic anisotropic nanoparticles composed of other shapes, like triangles and ellipsoids, long needles, which are also be referred to herein as wires. In a preferred embodiment of the invention, chains of nanospheres spheres approximate a needle.

By “unassembled” nanoparticles it is meant that nanoparticles in such a collection are not bound to each other through a physical force or chemical bond either directly (particle-particle) or indirectly through some intermediary (e.g. particle-cell-particle, particle-protein-particle, particle-analyte-particle).

By “assembled” nanoparticles it is meant that nanoparticles in such a collection are bound to at least one other nanoparticle through a physical force or chemical bond either directly (particle-particle) or indirectly through some intermediary (e.g. particle-cell-particle, particle-protein-particle, particle-analyte-particle). Examples of assembled nanoparticles useful in the methods of the present invention dimers, trimers and tetramers of plasmonic nanoparticles. Indeed a tetrahedron tetramer of unassembled plasmonic nanoparticles that have minimal if any local surface plasmon resonance when irradiated with light having a NIR wavelength—such a solid gold nanospheres having a diameter of less than about 100 nm are particularly preferred plasmonic material for use in the methods of the present invention.

The irradiation comprises light having a wavelength of light between about 200 nm and about 10,000 nm, a fluence of about 1 to about 100 joules/cm², a pulse width of about 1 femptosecond to about 1 second, and a repetition frequency of about 1 Hz to about 1 THz. 1 ns-200 ms pulse of light.

The process of the present invention delivers the plasmonic materials to the vicinity of the skin structures involved in the skin condition being treated. For instance, a plasmonic material could be delivered in the dermis or epidermis to a plurality of: hair follicles; sebaceous glands; sebaceous ducts; apocrine sweat glands; eccrine sweat glands; and/or oily glands.

In certain embodiments, this delivery is facilitated by application of mechanical agitation (e.g. massage), acoustic vibration in the range of 10 Hz-20 kHz, ultrasound, alternating suction and pressure, and microjets.

In one aspect, the invention generally provides methods of treating or ameliorating a follicular skin disease (e.g., acne) of a subject (e.g., human) The method involves topically applying a formulation containing a plasmonic material to a subject's skin; facilitating delivery of the compound to a hair follicle, sebaceous gland, sebaceous gland duct, or infundibulum of the skin by mechanical agitation, acoustic vibration, ultrasound, alternating suction and pressure, or microjets; and exposing the plasmonic material to energy activation, thereby treating the follicular skin disease.

In another aspect, the invention provides a method of treating or ameliorating a follicular skin disease of a subject, the method involving topically applying a formulation containing a plasmonic material, facilitating delivery of the compound to a hair follicle, sebaceous gland, sebaceous gland duct, or infundibulum of the skin by mechanical agitation, acoustic vibration, ultrasound, alternating suction and pressure, or microjets; and exposing the plasmonic material to energy activation, thereby treating the follicular disorder.

In another aspect, the invention provides a method of improving the appearance of enlarged pores in the skin of a subject, the method involving topically applying a formulation containing a plasmonic material to a subject's skin; facilitating delivery of the compound to a hair follicle, sebaceous gland, sebaceous gland duct, or infundibulum of the skin by mechanical agitation, acoustic vibration, ultrasound, alternating suction and pressure, or microjets; and exposing the plasmonic material to energy activation, thereby treating the follicular skin disease.

In another aspect, the invention provides a method for permanently removing lightly pigmented or thin hair of a subject, the method involving topically applying a light-absorbing compound to the skin of a subject, and exposing the compound to energy activation, thereby permanently removing the hair.

In another aspect, the invention provides a method for permanently removing lightly pigmented or thin hair of a subject, the method involving epilating hair from a follicle of the subject; topically applying a light-absorbing compound to the skin of a subject, and exposing the compound to energy activation, thereby permanently removing the hair. In one embodiment, the compound is a nanop article containing a silica core and a gold shell. In another embodiment, energy activation is accomplished with a pulsed laser light that delivers light energy at a wavelength that is absorbed by the particle. In another embodiment, the skin is prepared for the method by heating, by removing the follicular contents, and/or by epilation. In another embodiment, the topically applied plasmonic material is wiped from the skin prior to energy activation using acetone.

In another aspect, the invention provides a method of facilitating delivery of plasmonic material to a target volume within the skin of a subject, the method involving topically applying a formulation containing plasmonic material to a subject's skin to deliver the compound to a reservoir within the skin; facilitating delivery of the compound to a target volume within the skin of the subject by irradiating the skin with a first series of light pulses; and exposing the plasmonic material to a second series of light pulses to heat the compound and thermally damage the target volume to achieve a therapeutic effect. In a related approach, a train of low-energy laser pulses, 1 microsecond or less in pulse duration, preferably in the acoustic range for pulse repetition rate, is used to activate the particles. This activation violently ‘stirs’ the particles, some of which will be propelled from the infundibulum into the sebaceous glands.

In another aspect, the invention provides a method of facilitating delivery of plasmonic material to a target volume within the skin of a subject, the method involving topically applying a formulation containing plasmonic material to a subject's skin; facilitating delivery of the compound to a reservoir in the skin by mechanical agitation; facilitating delivery of the compound to a target volume within the skin by applying a train of low-energy laser pulses each pulse lasting for a microsecond or less to drive the material into the target volume; and exposing the plasmonic material to a second series of low-energy laser pulses to heat the compound and thermally damage the target volume to achieve a therapeutic effect.

In various embodiments of any of the above aspects or other aspects of the invention delineated herein, plasmonic material, nanoparticle, or nanoshell is coated with PEG. In other embodiments of the above aspects, the sub-micron particle is a nanoparticle containing a silica core and a gold shell, optionally coated with PEG. In certain embodiments, the nanoparticle or nanoshell is about 50-300 nm (e.g., 50, 75, 100, 125, 150, 175, 200, 300 nm).

The longest dimension of at least about 80% of said plasmonic sub-micron particles is less than about 800 nm; and the longest dimension of at least about 95% of said plasmonic sub-micron particles is greater than 100 nm.

In particular embodiments, the nanoparticle is coated with PEG. In embodiments of the invention, energy activation is accomplished with a pulsed laser light that delivers light energy at a wavelength that is absorbed by the particle. In other embodiments, the skin is prepared for the method by heating (e.g., to at least about 35-42° C.), by removing the follicular contents, and/or by epilation. In other embodiments, the follicular contents are removed by a method comprising contacting the follicle pore with adhesive polymers. In other embodiments, the topically applied plasmonic material is wiped from the skin prior to energy activation. In still other embodiments, the topically applied plasmonic material is wiped from the skin with acetone. In other embodiments, the follicular skin disease is acne vulgaris. In other embodiments, energy activation is carried out by irradiation of the skin with a laser. In other embodiments, the ultrasound energy has a frequency in the range of 20 kHz to 500 kHz. In other embodiments, the skin is heated before, during, or after topical application to about 42° C. or to a temperature sufficient to assist in follicular delivery. In other embodiments, the heating is accomplished via ultrasound. In other embodiments, the heating is not sufficient to cause pain, tissue damage, burns, or other heat-related effects in the skin. In other embodiments, the formulation contains a component (e.g., ethanol) having high volatility. In other embodiments, the formulation contains one or more of ethanol, isopropyl alcohol, propylene glycol, a surfactant, and/or isopropyl adipate. In other embodiments, the formulation contains hydroxypropylcellulose (HPC) and carboxymethyl cellulose (CMC). In other embodiments, the formulation contains any one or more of water, ethanol, propylene glycol, polysorbate 80, diisopropyl adipate, phospholipon, and thickening agents. In other embodiments, the formulation is a liposomal formulation.

### Composition.

In another aspect, the invention provides a composition comprising a cosmetically acceptable carrier and a plurality of plasmonic particles in an amount effective to induce thermomodulation in a target tissue region with which the composition is topically contacted. It is preferred that the carrier and plasmonic particles combination is a liquid with a low viscosity, i.e., a viscosity similar to that of water.

In one embodiment, the plasmonic nanoparticles are activated by exposure to energy delivered from a nonlinear excitation surface plasmon resonance source to the target tissue region. In another embodiment, the plasmonic nanoparticle comprises a metal, metallic composite, metal oxide, metallic salt, electric conductor, electric superconductor, electric semiconductor, dielectric, quantum dot or composite from a combination thereof. In yet another embodiment, a substantial amount of the plasmonic particles present in the composition comprise geometrically-tuned nanostructures.

In one embodiment, the plasmonic particles comprise any geometric shape currently known or to be created that absorb light and generate plasmon resonance at a desired wavelength, including nanoplates, solid nanoshells, hollow nanoshells, nanorods, nanorice, nanospheres, nanofibers, nanowires, nanopyramids, nanobipyramids, nanoprisms, nanostars or a combination thereof. In another embodiment, the plasmonic particles comprise silver, gold, nickel, copper, titanium, silicon, galadium, palladium, platinum, or chromium.

In one embodiment, the cosmetically acceptable carrier comprises an additive, a colorant, an emulsifier, a fragrance, a humectant, a polymerizable monomer, a stabilizer, a solvent, or a surfactant. In one particular embodiment, the surfactant is selected from the group consisting of sodium laureth 2-sulfate, sodium dodecyl sulfate, ammonium lauryl sulfate, sodium octech-1/deceth-1 sulfate, lipids, proteins, peptides or derivatives thereof. In another specific embodiment the surfactant is present in the composition in an amount between about 0.1 and about 10.0% weight-to-weight of the carrier.

In one embodiment, the solvent is selected from the group consisting of water, propylene glycol, alcohol, hydrocarbon, chloroform, acid, base, acetone, diethyl-ether, dimethyl sulfoxide, dimethylformamide, acetonitrile, tetrahydrofuran, dichloromethane, and ethylacetate.

In another embodiment, the composition comprises plasmonic particles that have an optical density of at least about 1 O.D. at one or more peak resonance wavelengths. A concentration of plasmonic particles of between about 10⁹ and 10¹⁴ particles per ml.

In yet another embodiment, the plasmonic particles comprise a hydrophilic or aliphatic coating, wherein the coating does not substantially adsorb to skin of a mammalian subject, and wherein the coating comprises polyethylene glycol, silica, silica-oxide, polyvinylpyrrolidone, polystyrene, a protein or a peptide.

In one embodiment, the thermomodulation comprises damage, ablation, lysis, denaturation, deactivation, activation, induction of inflammation, activation of heat shock proteins, perturbation of cell-signaling or disruption to the cell microenvironment in the target tissue region.

In another embodiment, the target tissue region comprises a sebaceous gland, a component of a sebaceous gland, a sebocyte, a component of a sebocyte, sebum, or hair follicle infundibulum. In a specific embodiment, the target tissue region comprises a bulge, a bulb, a stem cell, a stem cell niche, a dermal papilla, a cortex, a cuticle, a hair sheath, a medulla, a pylori muscle, a Huxley layer, or a Henle layer.

In another aspect, the invention provides a method for performing targeted ablation of a tissue to treat a mammalian subject in need thereof, comprising the steps of i) topically administering to a skin surface of the subject a composition of the invention as described above; ii) providing penetration means to redistribute the plasmonic particles from the skin surface to a component of dermal tissue; and iii) causing irradiation of the skin surface by light.

In one embodiment, the light source comprises excitation of mercury, xenon, deuterium, or a metal-halide, phosphorescence, incandescence, luminescence, light emitting diode, or sunlight.

In another embodiment, the penetration means comprises high frequency ultrasound, low frequency ultrasound, massage, iontophoresis, high pressure air flow, high pressure liquid flow, vacuum, pre-treatment with fractionated photothermolysis or dermabrasion, or a combination thereof.

In yet another embodiment, the irradiation comprises light having a wavelength of light between about 200 nm and about 10,000 nm, a fluence of about 1 to about 100 joules/cm², a pulse width of about 1 femptosecond to about 1 second, and a repetition frequency of about 1 Hz to about 1 THz.

In another aspect, the invention provides a composition comprising a cosmetically acceptable carrier, an effective amount of sodium dodecyl sulfate, and a plurality of plasmonic nanoparticles in an amount effective to induce thermal damage in a target tissue region with which the composition is topically contacted, wherein the nanoparticles have an optical density of at least about 1 O.D. at a resonance wavelength of about 810 nanometers or 1064 nanometers, wherein the plasmonic particles comprise a silica coating from about 5 to about 35 nanometers, wherein the acceptable carrier comprises water and propylene glycol.

In still another aspect, the invention provides a system for laser ablation of hair or treatment of acne comprising a composition of the invention as described above and a source of plasmonic energy suitable for application to the human skin.

The invention provides compositions, methods and systems for treating follicular skin diseases. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “plasmonic material” is meant metamaterials that exploit surface plasmons to achieve optical properties. Surface plasmons are produced from the interaction of light with metal-dielectric materials. Under specific conditions, the incident light couples with the surface plasmons to create self-sustaining, propagating electromagnetic waves known as surface plasmon polaritons (SPPs).

By “ameliorate” is meant decrease, suppress, attenuate, diminish, arrest, or stabilize the development or progression of a skin disease or condition. One exemplary skin condition is acne vulgaris.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “energy activation” is meant stimulation by an energy source that causes thermal or chemical activity. Energy activation may be by any energy source known in the art. Exemplary energy sources include a laser, ultrasound, acoustic source, flashlamp, ultraviolet light, an electromagnetic source, microwaves, or infrared light. An energy absorbing compound absorbs the energy and become thermally or chemically active.

As used herein, “obtaining” as in “obtaining an agent” includes synthesizing, purchasing, or otherwise acquiring the agent.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a energy activatable material of the present invention within or to the subject such that it can performs its intended function. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Preferred carriers include those which are capable of entering a pore by surface action and solvent transport such that the energy activatable material is carried into or about the pore, e.g., into the sebaceous gland, to the plug, into the infundibulum and/or into the sebaceous gland and infundibulum.

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, or feline. As used herein, the term “sub-micron particle” refers to material in the largest dimension of at least about 80% of said material is less than about 800 nm. Preferably the largest dimension of at least about 80% of the sub-micron particles of the present invention is at least about 100 nm, but less than about 650 nm. It is further preferred that the largest dimension of at least about 80% of the sub-micron particles of the present invention is at least about 120 nm, but less than about 500 nm.

The sub-micron particles of the present invention are plasmonic in that these particles have free electrons that are excited by the electric component of infrared light to have collective oscillations. It is preferred that the sub-micron particles of the present invention have a peak absorption peak for infrared light with a wavelength of between 720 and 1200 nm.

For a composition of the present invention, it is preferred that at least about 68% of the sub-micron particle's absorption spectra peak is between 720 and 1200 nm. It is believe that for many nanoparticles, a substantial portion of the “absorption” in the wavelengths corresponding to either tail of an absorption spectra peak is due to scattering, and not absorption. It is further believed that scattering results in very little, if any, plasmonic resonance.

Plasmonic materials have been created from many different materials. Typically, plasmonic materials comprise a metal, but plasmonic materials have been formed from other materials. Silver, gold, nickel, copper, titanium, silicon, gallium, palladium, platinum, chromium, and titanium nitride are typical examples of materials used to create a plasmonic material.

There are numerous factors that impact the peak absorption wavelength a plasmonic particle. For instance, for silver nanoparticles, “[a]s the particle size increases from 10 to 100 nm, the absorbance peak (lambda max) increases from 400 nm to 500 nm . . . .” http://www.cytodiagnostics.com/store/pc/Silver-Nanoparticle-Properties-d11.htm last viewed on Sep. 24, 2015. While gold nanoparticles generally absorb at longer wavelengths that silver nanop articles of the same size, for gold nanop articles, as the particle size increases from 10 to 100 nm, the absorbance peak increase from 500 nm to 600 nm. See http://nanocomposix.com/pages/gold-nanoparticles-optical-properties last viewed on Sep. 24, 2015. Thus neither gold nor silver spherical nanoparticles having a diameter of 100 nm or less have a peak absorption peak for infrared light with a wavelength of between 720 and 1200 nm.

Even larger gold and silver spherical nanoparticles generally do have a peak absorption peak for infrared light with a wavelength of between 720 and 1200 nm. http://nanocompsix.com/pages/plasmonic-nanoparticles last viewed Sep. 25, 2015.

There are other ways known in the art of creating sub-micron particles that have a peak absorption peak for infrared light with a wavelength of between 720 and 1200 nm. For instance, a metal coated silica nanoparticle, depending upon the size of the silica core and the metal coating, may have a peak absorption peak for infrared light with a wavelength of between 720 and 1200 nm. As an example, a 120 nm diameter silica nanosphere coated with a 15 nm gold shell, has a peak absorption of about 900 nm. Prashant K. Jain, Kyeong Seok Lee, Ivan H. El-Sayed, and Mostafa A. El-Sayed, Calculated Absorption and Scattering Properties of Gold Nanoparticles of Different Size, Shape, and Composition: Applications in Biological Imaging and Biomedicine, 110 J. Phys. Chem. B 7238 (2006).

Additionally, it has been observed that changing the geometry of the sub-micron particle changes the wavelength of peak absorption. For instance, depending upon the aspect ratio (length to diameter), gold nanorods have a wavelength of peak absorption including 660 nm, 800 nm, and 980 nm. See http://nanocomposix.com/collections/gold-nanorods last viewed Sep. 25, 2015. Specifically, 50 nm by 19 nm gold rods (i.e., having a 2.7 aspect ratio) have a peak absorption wavelength of 660 nm, just outside of the 720 to 1200 nm range. However, 70 nm by 19 nm gold rods (i.e., having a 3.6 aspect ratio) and 70 nm by 12 nm gold rods (i.e., having a 6.1 aspect ratio) have peak absorption wavelengths of 800 nm and 980 nm respectively.

Another non-spherical geometry that has been observed are nanoplates. These structures generally are: disk-like, approximate a triangular prism, or a prism having a shape intermediate between a circle (i.e., disk like) and a triangle. See http://nanocomposix.com/collections/silver-nanoplates/products/550-nm-resonant-silver-nanoplates last viewed on Sep. 25, 2015. Silver nanoplates having a diameter of 40 to 60 nm and a thickness of 10 nm (i.e., an aspect ratio of 4 to 6) are reported to have a peak absorption wavelength of 550 nm.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 20%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

The invention features compositions comprising light/energy absorbing compounds and methods that are useful for their topical delivery to a target (e.g., a follicle, follicular infundibulum, sebaceous gland) for the treatment of a follicular disease.

### Follicular Disease Pathogenesis

Sebaceous glands are components of the pilosebaceous unit. They are located throughout the body, especially on the face and upper trunk, and produce sebum, a lipid-rich secretion that coats the hair and the epidermal surface. Sebaceous glands are involved in the pathogenesis of several diseases, the most frequent one being acne vulgaris. Acne is a multifactorial disease characterized by the occlusion of follicles by plugs made out of abnormally shed keratinocytes of the infundibulum (upper portion of the hair follicle) in the setting of excess sebum production by hyperactive sebaceous glands.

The infundibulum is an important site in the pathogenesis of many follicular diseases (e.g., acne). There is evidence that abnormal proliferation and desquamation of infundibular keratinocytes leads to the formation of microcomedones and, subsequently, to clinically visible follicular “plugs” or comedones. Because the architecture of the infundibulum is important in the pathogenesis of acne, the selective destruction of this portion of the follicle through energy activatable material-assisted energy, e.g., laser, targeting eliminates or reduces the site of pathology.

### Topical Delivery of Plasmonic Materials

The invention provides delivery of plasmonic materials via topical application into skin appendages of the follicle, specifically follicular infundibulum and the sebaceous gland. In one embodiment, such compounds are useful for the treatment of follicular diseases, such as acne (e.g., acne vulgaris). The introduction of plasmonic materials into sebaceous glands followed by exposure to energy (light) with a wavelength that corresponds to a wavelength at which a local surface plasmon resonance occurs in the plasmonic material will increase the local absorption of light in tissue and lead to selective thermal damage of sebaceous glands.

### Skin Preparation

If desired, the skin is prepared by one or a combination of the following methods. Delivery of plasmonic material may be facilitated by epilation of hair, which is performed prior to topical application of the plasmonic material.

Optionally, the skin is degreased prior to application of the plasmonic material. For example, acetone wipes are used prior to application of sebashells to degrease the skin, especially to remove the sebum and follicular contents.

For certain subjects, delivery may be facilitated by reducing or clearing clogged follicles prior to application of the light absorbing material. Such clearing can enhance the delivery of the nanoshells. The follicles, especially in acne prone patients, are clogged by shed keratinocytes, sebum, and bacteria P. acnes. The follicle can be emptied by application of vacuum. Other methods are cyanoacrylate stripping, strips with components such as Polyquaternium 37 (e.g., Biore pore removal strips). The polymers flow into the follicle and dry over time. When the dry polymer film is pulled out, the follicular contents are pulled out, emptying the follicle.

Optionally, the skin may be heated prior to application of the plasmonic material. Heating reduces the viscosity of the sebum and may liquefy components of the sebum. This can facilitate delivery of plasmonic material (e.g., formulated as nanoshells) to the follicle.

### Topical Delivery of Plasmonic Material

Plasmonic material are topically applied to the skin following any desired preparation. The topically applied formulations containing the plasmonic materials may comprise ethanol, propylene glycol, surfactants, and acetone. Such additional components facilitate delivery into the follicle.

Delivery of plasmonic material is facilitated by application of mechanical agitation, such as massage, acoustic vibration in the range of 10 Hz-20 kHz, ultrasound, alternating suction and pressure, and jets. In one embodiment, plasmonic material are delivered as nanoparticles, such as nanoshells or nanorods that absorb light in the visible and the near-IR region of the electromagnetic spectrum. In another embodiment, plasmonic material are quantum dots. Preferably, the plasmonic material are formulated for topical delivery in a form that facilitates follicular delivery. In one embodiment, such formulations comprise water, ethanol, isopropyl alcohol, propylene glycol, surfactants, and isopropyl adipate and related compounds.

### Ultrasound Facilitated Delivery

Ultrasound has been used to achieve transdermal delivery of compounds into the body. Ultrasound appears to generate shock-waves and micro-jets resulting from bubble cavitation that causes the formation of channels in the skin, which provide for the transport of molecules of interest. Previous efforts have been directed toward the delivery of the compounds through the stratum corneum. Small molecules, for example, with sizes less than 5 nm, can be delivered through the stratum corneum. The delivery rate through the stratum corneum goes down significantly as particle size increases. For example, for particles with size of 50 nm and higher, the delivery rate through the stratum corneum is very low. However, this size is still much smaller than the pore opening and the infundibulum of a follicle. For example, 150 nm size silica-core and gold shell structures are being used that are much smaller than the infundibular diameter while showing low deposition in skin through the stratum corneum.

These findings provide the basis of acne treatment in which the infundibulo-sebaceous unit is selectively targeted for first delivery of light absorbing material of appropriate size and then selective thermal damage to the unit with pulsed laser irradiation. Here, ultrasound specifically facilitates the delivery of plasmonic material into the follicular structure. The shock waves, microjet formation, and streaming deliver the light absorbing particles into the follicular infundibulum and the associated sebaceous gland duct and the sebaceous gland.

Ultrasound is often be accompanied by heating of the target organ, skin. Some heating, for example, up to about 42° C. may help in follicular delivery. However, excessive heating is undesirable, causing pain, tissue damage, and burns. In one embodiment, excessive heating can be avoided by cooling the skin, for example In another embodiment, the topically applied formulation or a coupling gel can be pre- or parallel-cooled. A low duty cycle with repeated ultrasound pulse bursts can also be used to avoid excessive heating, where during the off-time, the body cools the skin that is being subjected to ultrasound energy.

Acoustic cavitation is often an effect observed with ultrasound in liquids. In acoustic cavitation, a sound wave imposes a sinusoidally varying pressure upon existing cavities in solution. During the negative pressure cycle, the liquid is pulled apart at ‘weak spots’. Such weak spots can be either pre-existing bubbles or solid nucleation sites. In one embodiment, a bubble is formed which grows until it reaches a critical size known as its resonance size (Leong et al., Acoustics Australia, 2011—acoustics.asn.au, THE FUNDAMENTALS OF POWER ULTRASOUND—A REVIEW, p 54-63). According to Mitragotri (Biophys J. 2003; 85(6): 3502-3512), the spherical collapse of bubbles yields high pressure cores that emit shock waves with amplitudes exceeding 10 kbar (Pecha and Gompf, Phys. Rev. Lett. 2000; 84:1328-1330). Also, an aspherical collapse of bubbles near boundaries, such as skin yields microjets with velocities on the order of 100 m/s (Popinet and Zaleski, 2002; J. Fluid. Mech. 464:137-163). Such bubble-collapse phenomena can assist in delivery of materials into skin appendages, such as hair and sebaceous follicles. Thus, the invention provides methods for optimizing bubble size before collapse to promote efficient delivery of plasmonic material into the intended target (e.g., sebaceous glands, hair follicles).

The resonance size of the bubble depends on the frequency used to generate the bubble. A simple, approximate relation between resonance and bubble diameter is given by F (in Hz)×D (in m)=6 m.Hz, where F is the frequency in Hz and D is the bubble diameter (size) in m. In practice, the diameter is usually smaller than the diameter predicted by this equation due to the nonlinear nature of the bubble pulsation.

For efficient delivery into the follicles with cavitation bubbles, there is an optimal cavitation bubble size range. Strong cavitational shock waves are needed, which are generated with relatively large bubbles. However, if the bubble size is too large, it produces strong shock waves, which may compress the skin, reducing the pore size, and reducing efficient delivery to a target (e.g., sebaceous gland, follicle). For example, if the bubble size is much larger than the follicle opening, the resulting shock waves compress not only the pore opening, but also the skin surrounding the pore opening. This inhibits efficient delivery into the follicle opening. Desirably, bubble sizes should be about the same size as the target pore. Typical pore sizes of follicles on human skin are estimated to be in the range of 12-300 microns. Thus, the preferred ultrasound frequency range is 20 kHz to 500 kHz. The desired power density is estimated to be in the range of 0.5-10 W/cm 2. This is sufficient to generate cavitation bubbles in the desired size range.

### Energy (Light) Activation

After the topical application and facilitated delivery (e.g., by mechanical agitation, ultrasound), the top of the skin is wiped off to remove the residual light absorbing material. This is followed by energy (light) irradiation. The light is absorbed by the material inside the follicle or sebaceous gland leading to localized heating. The light source depends on the absorber used. For example, for nanoshells that have broad absorption spectrum tuned to 800 nm resonance wavelength, sources of light such as 800-nm, 755-nm, 1,064-nm or intense pulsed light (IPL) with proper filtering can be used. Such pulsed laser irradiation leads to thermal damage to the tissue surrounding the material. Damage to infundibular follicular stem cells and/or sebaceous glands leads to improvement in the follicular conditions, such as acne. Such methods can be used not only for particulates in suspensions but for small dissolved molecules in solution as well.

Suitable energy sources include light-emitting diodes, incandescent lamps, xenon arc lamps, lasers or sunlight. Suitable examples of continuous wave apparatus include, for example, diodes. Suitable flash lamps include, for example pulse dye lasers and Alexandrite lasers. Representative lasers having wavelengths strongly absorbed by chromophores, e.g., laser sensitive dyes, within the epidermis and infundibulum but not sebaceous gland, include the short-pulsed red dye laser (504 and 510 nm), the copper vapor laser (511 nm) and the Q-switched neodymium (Nd):YAG laser having a wavelength of 1064 nm that can also be frequency doubled using a potassium diphosphate crystal to produce visible green light having a wavelength of 532 nm. In the present process, selective photoactivation is employed whereby an energy (light) source, e.g., a laser, is matched with a wave-length to the absorption spectrum of the selected plasmonic material.

It is easier to achieve a high concentration of the light absorbing material in the infundibulum than the sebaceous duct and the gland, which provide a higher resistance to material transport. The follicle including the sebaceous gland can be irreversibly damaged just relying on light absorption principally but the material in the infundibulum. This is mediated through damage to the keratinocytes in the follicular epithelium. Also, with higher energy pulses can be used to extend the thermal damage to include the stem cells in the outer root sheath, the bulge, as well as the outside periphery of the sebaceous glands. However, such high energy should not lead to undesired side effects. Such side effects can be mitigated by use of cooling of the epidermis and also use of longer pulse durations, on the order of several milliseconds, extending up to 1,000 ms.

Thermal alteration of the infundibulum itself with only limited involvement of sebaceous glands may improve acne. Appearance of enlarged pores on the face is a common issue for many This is typically due to enlarged sebaceous glands, enlarged infundibulum, as well as enlarged pore opening. Heating of tissue, especially collagen, shrinks the tissue. The delivery of nanoshells and thermal targeting of the same in the infundibulo-sebaceous unit that includes the upper, lower infundibulum, as well as the sebaceous gland, will improve the appearance of enlarged pores.

### Formulations of Plasmonic Materials

The invention provides compositions comprising plasmonic materials for topical delivery. In one embodiment, a compound of the invention comprises a silica core and a gold shell (150 nm). In another embodiment, nanoshells used are composed of a 120 nm diameter silica core with a 15 micron thick gold shell, giving a total diameter of 150 nm. The nanoshell is covered by a 5,000 MW PEG layer. The PEG layer prevents and/or reduces nanoshell aggregation, thereby increasing the nanoshell suspensions stability and shelf-life.

Nanoparticles of the invention exhibit Surface Plasmon Resonance, such that Incident light induces optical resonance of surface plasmons (oscillating electrons) in the metal. The Wavelength of peak absorption can be “tuned” to the near-infrared (IR) portion of the electromagnetic spectrum. The submicron size of these nanoparticles allows their entry into the infundibulum, sebaceous duct and sebaceous gland of the epidermis, and minimizes their penetration of the stratum corneum. In particular embodiment, selective transfollicular penetration of nanoparticles about 150-350 nm in diameter is achieved.

If desired, light/energy absorbing compounds are provided in vehicles formulated for topical delivery. In one embodiment, a compound of the invention is formulated with agents that enhance follicular delivery, including but not limited to, one or more of ethanol, isopropyl alcohol, propylene glycols, surfactants such as polysorbate 80, Phospholipon 90, polyethylene glycol 400, and isopropyl adipate. In other embodiments, a compound of the invention is formulated with one or more thickening agents, including but not limited to, hydroxypropylcellulose (HPC) and carboxymethyl cellulose (CMC), to enhance handling of the formulations.

Wetting agents, emulsifiers and lubricants, such as sodium lauryl sulfate and magnesium stearate, as well as coloring agents, release agents, coating agents, sweetening and perfuming agents, preservatives and antioxidants can also be present in the compositions.

Liquid dosage forms for topical administration of the compounds of the invention include pharmaceutically acceptable emulsions, microemulsions, solutions, creams, lotions, ointments, suspensions and syrups. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, peach, almond and sesame oils), glycerol, tetrahydrofuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. The term “cream” is art recognized and is intended to include semi-solid emulsion systems which contain both an oil and water. Oil in water creams are water miscible and are well absorbed into the skin, Aqueous Cream BP. Water in oil (oily) creams are immiscible with water and, therefore, more difficult to remove from the skin. These creams are emollients, lubricate and moisturize, e.g., Oily Cream BP. Both systems require the addition of either a natural or a synthetic surfactant or emulsifier.

The term “ointment” is art recognized and is intended to include those systems which have oil or grease as their continuous phase. Ointments are semi-solid anhydrous substances and are occlusive, emollient and protective. Ointments restrict transepidermal water loss and are therefore hydrating and moisturizing. Ointments can be divided into two main groups—fatty, e.g., White soft paraffin (petrolatum, Vaseline), and water soluble, e.g., Macrogol (polyethylene glycol) Ointment BP. The term “lotion” is art recognized and is intended to include those solutions typically used in dermatological applications. The term “gel” is art recognized and is intended to include semi-solid permutations gelled with high molecular weight polymers, e.g., carboxypolymethylene (Carbomer BP) or methylcellulose, and can be regarded as semi-plastic aqueous lotions. They are typically non-greasy, water miscible, easy to apply and wash off, and are especially suitable for treating hairy parts of the body.

### Subject Monitoring

The disease state or treatment of a subject having a skin disease or disorder can be monitored during treatment with a composition or method of the invention. Such monitoring may be useful, for example, in assessing the efficacy of a particular agent or treatment regimen in a patient. Therapeutics that promote skin health or that enhance the appearance of skin are taken as particularly useful in the invention.

### Kits

The invention provides kits for the treatment or prevention of a skin disease or disorder, or symptoms thereof. In one embodiment, the kit includes a pharmaceutical pack comprising an effective amount of a light/energy absorbing compound (e.g., a nanoshell having a silica core and a gold shell (150 nm)). Preferably, the compositions are present in unit dosage form. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention or combinations thereof are provided together with instructions for administering them to a subject having or at risk of developing a skin disease or disorder. The instructions will generally include information about the use of the compounds for the treatment or prevention of a skin disease or disorder. In other embodiments, the instructions include at least one of the following: description of the compound or combination of compounds; dosage schedule and administration for treatment of a skin condition associated with acne, dermatitis, psoriasis, or any other skin condition characterized by inflammation or a bacterial infection, or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The following examples are provided to illustrate the invention, not to limit it. those skilled in the art will understand that the specific constructions provided below may be changed in numerous ways, consistent with the above described invention while retaining the critical properties of the compounds or combinations thereof.

### Laser Hair Removal

The invention features compositions and methods that are useful for laser hair removal, particularly in light colored hair. In laser hair removal, a specific wavelength of light and pulse duration is used to obtain optimal effect on a targeted tissue with minimal effect on surrounding tissue. Lasers can cause localized damage to a hair follicle by selectively heating melanin, which is a dark target material, while not heating the rest of the skin. Because the laser targets melanin, light colored hair, gray hair, and fine or thin hair, which has reduced levels of melanin, is not effectively targeted by existing laser hair removal methods. Efforts have been made to deliver various material, such as carbon particles, extracts from squid ink, known commercially as meladine, or dyes into the follicle. These methods have been largely ineffective.

The present invention provides plasmonic materials in a suspension form that is topically applied after skin preparation as delineated herein above. In particular, the skin is prepared by epilation of the hair shaft and plasmonic material are delivered to the hair follicle. Preferably, the formulation is optimized for follicular delivery with mechanical agitation for a certain period of time. After wiping off the formulation from the top of the skin, laser irradiation is performed, preferably with surface cooling. The laser is pulsed, with pulse duration approximately 0.5 ms-400 ms using a wavelength that is absorbed by the nanoshells. This method will permanently remove unpigmented or lightly pigmented hair by destroying the stem cells and other apparatus of hair growth which reside in the bulge and the bulb area of the follicle.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Preparation of Assembled Nanoparticles

Assembled nanoparticles can be prepared from monodisperse silica microspheres, typically such silica microspheres having a diameter of between about 20 nm and 400 nm are commercially available from, for instance, nanoComposix, Inc. of San Diego, Calif. The silica microspheres are then functionalized via, for example, amination and silanization. In the next step a gold colloidal solution dispersion is prepared. The functionalized silica microspheres are blended with the gold colloid, which will yield “seeds” of silica microspheres coated with gold patches. These silica microspheres coated with gold patches seeds are mixed with a potassium gold-plating solution. This process produces plasmonic at a low concentration, e.g., OD of 1-2 (about 2.7×10⁹ particles/ml at an OD of 1). The particles could be concentrated by either tangential flow filtration or centrifugation to obtain a higher concentration plasmonic nanoparticle dispersion, perhaps OD of 1,200, in water. A substantial percent of the nanoparticles prepared by this route will be assembled.

To prepare a composition for topical application, the plasmonic nanoparticle dispersion is diluted with ethanol, diisopropyl adipate, and a surfactant.

The diluted plasmonic nanoparticle dispersion may be stored in glass vials until used for treatment.

### Example 2

### Alternative Preparation of Assembled Nanoparticles

Commercially available plasmonic nanoparticles that produce a local surface plasmon when irradiated with light having a wavelength of less than about 600 nm, but not in response to light having a longer wavelength (for instance silver nanospheres having a diameter of between about 10 nm and 30 nm from , nanoComposix, Inc. of San Diego, Calif.) when treated to form tetramers resonate in response to light with a wavelength between about 750 nm and 1200 nm.

### Example 3

### Plasmonic Nanoparticle Treatment of Skin Infections

Opsonized plasmonic sub-micron particles are dispersed in a dermatologically acceptable carrier at a concentration having an O.D. of between about 1 and 250. In a first formulation, the plasmonic sub-micron particles are opsonized by functionalizing said particles with a glycosaminoglycan such as keratan sulfate, or chondroitin sulfate.

In an alternative embodiment, the plasmonic sub-micron particles are gold nanorods (for instance, a 10 nm thick and 40 nm long gold nanorods) are embedded in cholesteric liquid crystals.

The opsonized plasmonic sub-micron particles are then applied to a microbiologically infected skin surface. The microorganism causing the infection, ingests the opsonized plasmonic sub-micron particles. Thereafter, the infected skin surface to which the opsonized plasmonic sub-micron particles were applied is irradiated with light having a wavelength at which the particles generate a local surface plasmon. These surface plasmons heat the interior of the infecting microorganism causing it to die.

### Example 4

### Hyperhidrosis Treatment

A dispersion of plasmonic material is applied to a skin surface having a plurality of sweat glands. Using a mechanical vibrator, the plasmonic nanoparticles are moved from the skin surface into a plurality of said sweat glands. The dispersion that remains visible on the skin surface is removed. Thereafter the plasmonic material in the sweat glands were irradiated with NIR light and they generated local surface plasmons. These surface plasmons heated the interior of the sweat glands and thermally damaged these glands. Thereafter, the damaged sweat glands produced less sweat.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

We claim:

1. A method of treating a skin condition comprising:
Obtaining a composition of plasmonic sub-micron particles wherein (1) (a) the longest dimension of at least about 80% of said plasmonic sub-micron particles is less than about 800 nm; and (b) the longest dimension of at least about 95% of said plasmonic sub-micron particles is greater than 100 nm; and (2) said plasmonic sub-micron particles are in a dermatologically acceptable carrier;
Said composition has a concentration of plasmonic particles of between about 10⁹ and 10¹⁴ particles per ml,
Said plasmonic sub-micron particles generating a surface plasmon when irradiated with light having a wavelength between about 750 and 1200 nm;
Said plasmonic sub-micron particles comprising silver, gold, nickel, copper, titanium, palladium, platinum, chromium, or titanium nitride.
Applying said plasmonic sub-micron particle composition to a skin surface having a condition to be treated;
Moving said plasmonic sub-micron particles in said applied composition from said skin surface into a plurality of epidermal appendages;
Removing said plasmonic sub-micron particles remaining on said skin surface after a portion of said plasmonic sub-micron particles have been moved into a plurality of epidermal appendages;
Irradiating said plasmonic sub-micron particles in said plurality of epidermal appendages with a 1 ns-200 ms pulse of light having a wavelength between about 750 and 1200 nm.

2. The method of claim 1 in which a majority of said plasmonic sub-micron particles further comprise plasmonic nanoparticles selected from the group consisting of nanoplates, solid nanoshells, hollow nanoshells, nanorods, nanorice, nanospheres, nanofibers, nanowires, nanopyramids, nanobipyramids, nanoprisms, nanostars and combinations thereof.

3. A method of treating a skin condition comprising:
Applying a composition of composite plasmonic nanoparticles to a skin surface having a condition to be treated;
Said composition having a concentration of composite plasmonic particles of between about 10⁹ and 10¹⁴ nanoparticles per ml,
Said composite particles comprising assembled plasmonic nanoparticles and having a size of about 100 to 500 nm,
Said composite plasmonic particles generating a surface plasmon when irradiated with light having a wavelength between about 700 and 1200 nm;
Said composite plasmonic particles comprising silver, gold, nickel, copper, titanium, silicon, gallium, palladium, platinum, chromium, or titanium nitride.

4. A method of treating a skin condition comprising:
Obtaining a composition of composite plasmonic nanoparticles;
Said composition having a concentration of composite plasmonic particles of between about 10⁹ and 10¹⁴ nanoparticles per ml,
Said composite particles comprising assembled plasmonic nanoparticles and having a size of about 100 to 500 nm,
Said composite plasmonic particles generating a surface plasmon when irradiated with light having a wavelength between about 700 and 1200 nm;
Said composite plasmonic particles comprising silver, gold, nickel, copper, titanium, palladium, platinum, chromium, or titanium nitride.

5. A method of treating a skin condition in need of treatment, said method comprising:
applying a composition of plasmonic particles to a skin surface,
moving said plasmonic particles from the skin surface into a plurality of openings in said skin surface,
removing said composition from said skin surface after moving said particles into skin openings, and
irradiating said particles in said skin openings with light having a wavelength between about 700 and 1200 nm to induce surface plasmons in said plasmonic particles,
Wherein said plasmonic particles are composite particles, at least about 95% of which have a size of at least 100 nn and at least about 90% of which have a size of less than 500 nm.

6. A method of treating a skin condition in need of treatment, said method comprising:
Obtaining a suspension of plasmonic sub-micron particles dispersed in a dermatologically acceptable carrier, said plasmonic sub-micron particles have a longest dimension and (a) the longest dimension of at least about 80% of said plasmonic sub-micron particles is less than about 800 nm and (b) the longest dimension of at least about 95% of said plasmonic sub-micron particles is more than 100 nm;
The exterior of said plasmonic sub-micron particles is a coating that comprises at least one member of the group consisting of polyethylene glycol (PEG), silica, silica-oxide, polyvinylpyrrolidone, polystyrene, silica, silver, polyvinylpyrrolidone (PVP), cetyl trimethylammonium bromide (CTAB), citrate, lipoic acid, short chain polyethylenimine (PI) and branched polyethylenimine, reduced graphene oxide, a protein, a peptide, and a glycosaminoglycan;
Said composition has a concentration of plasmonic particles of between about 10⁹ and 10¹⁴ particles per ml,
Said plasmonic sub-micron particles generating a surface plasmon when irradiated with light having a wavelength between about 750 and 1200 nm;
Said plasmonic sub-micron particles comprising silver, gold, nickel, copper, titanium, palladium, platinum, chromium, or titanium nitride.
Applying said plasmonic sub-micron particle composition to a skin surface having a condition to be treated;
Moving said plasmonic sub-micron particles in said applied composition from said skin surface into a plurality of epidermal appendages;
Removing said plasmonic sub-micron particles remaining on said skin surface after a portion of said plasmonic sub-micron particles have been moved into a plurality of epidermal appendages;
Irradiating said plasmonic sub-micron particles in said plurality of epidermal appendages with a 1 ns-200 ms pulse of light having a wavelength between about 750 and 1200 nm.

7. A method of treating a skin condition in need of treatment, said method comprising:
Obtaining a suspension of opsonized plasmonic sub-micron particles dispersed in a dermatologically acceptable carrier;
Said opsonized plasmonic sub-micron particles comprising a conductive metal and an exterior coating;
Said conductive metal comprising at least one metal selected from the group consisting of silver, gold, nickel, copper, titanium, palladium, platinum, chromium, and titanium nitride;
Said exterior coating comprising at least one member of the group consisting of polyethylene glycol (PEG), silica, silica-oxide, polyvinylpyrrolidone, polystyrene, silica, silver, polyvinylpyrrolidone (PVP), cetyl trimethylammonium bromide (CTAB), citrate, lipoic acid, short chain polyethylenimine (PI) and branched polyethylenimine, reduced graphene oxide, a protein, a peptide, and a glycosaminoglycan;
Said suspension having a concentration of opsonized plasmonic sub-micron particles of between about 10⁹ and 10¹⁴ particles per ml,
Applying said opsonized plasmonic sub-micron particle composition as an aerosol to a skin surface having a condition to be treated;
Irradiating said opsonized plasmonic sub-micron particles applied to said skin surface with a 1 ns-200 ms pulse of light having a wavelength between about 750 and 1200 nm.
